(19) United States
(12) Patent Application Publication
(10) Pub. No.: US 2026/0008744 A1
(43) Pub. Date: Jan. 8, 2026

(54) METHOD FOR (TRANS)ESTERIFICATION OF (METH)ACRYLATE COMPOUNDS USING STRONG BASIC CATALYSTS

(71) Applicant: Evonik Operations GmbH, Essen (DE)

(72) Inventors: Marcel Treskow, Darmstadt (DE); Kevin Tyrone Lackey, Fairhope, AL (US); Daniel Barnett, Gulf Shores, AL (US)

(73) Assignee: Evonik Operations GmbH, Essen (DE)

(21) Appl. No.: 18/873,617

(22) PCT Filed: Jun. 14, 2023
(86) PCT No.: PCT/EP2023/065938

§ 371 Date: Dec. 10, 2024

(30) Foreign Application Priority Data
Jun. 15, 2022 (EP) 22179164.3
Jun. 27, 2022 (EP) 22181206.8

(51) Int. Cl. C07C 69/602; B01J 23/04; B01J 31/16; C07C 31/20; C07C 67/02; C07C 69/54; C07C 69/732

(52) U.S. Cl. CPC C07C 69/602; B01J 23/04; B01J 31/1616; C07C 31/202; C07C 67/02; C07C 69/54; C07C 69/732; B01J 2231/49

(57) ABSTRACT

A method prepares an alkyl (meth)acrylate product by a (trans)esterification reaction of a reaction mixture in a reactor system. The reactor system has a reaction chamber with the reaction mixture, a feed line, a reboiler, a column with a column head, a vapor transfer line, a condenser, a reflux tank, a reflux line, a distillate take off line, and a receiver vessel.

### FIELD OF THE INVENTION

The invention relates to the field of discontinuous (trans)esterification of (meth)acrylate compounds with an alcohol using a strong basic catalyst to produce a new target product with separation of the side product methanol.

### BACKGROUND OF THE INVENTION

The production of special methacrylates can be efficiently achieved by the transesterification of methyl methacrylate (MMA) and an alcohol as starting material as well as by using a catalyst. Titanium(IV) alkoxide catalysts are widely used due to their selectivity and commercial availability. However, titanium(IV) catalysts deactivate readily and irreversibly with chelating alcohols. In particular, 1,2- or 1,3-difunctional raw materials rapidly deactivate said catalysts.

This can be circumvented for disubstituted 1,3-raw materials by zirconia catalysts such as Zr(acac)₄, that is Zr(C₅H₇O₂)₄, with higher selectivity. However, similar to Ti(IV) alkoxide, Zr(acac)₄ catalyst generally cannot catalyze the reaction of a 1,2-difunctional alcohol substrate such as ethylene glycol, glycerol, 1,2-propandiol or vicinal diols.

For this reason, the method of choice is to use a strong basic catalyst, as far as vicinal diols are subjected to transesterification with MMA or existing contaminants in the raw materials. These strong basic catalysts include oxide derivatives of alkali and alkaline earth metals or even some transition metal oxides or strong alkaline ion exchange resins. All these catalysts produce alkoxides of the raw material alcohol, which are transesterified to methanolate, which is released to methanol by proton exchange with a further raw material alcohol.

Among the strong basic catalysts, calcium oxides and hydroxides are characterized by their safety, recognized up to food suitability, and the cheapest raw material costs. However, they are not as fast as another group of catalysts, such as alkoxides. Theoretically, all alkoxides are readily suitable for the generation of the catalytic species of an alkoxide. However, only lithium salts have the unique ability to prevent anionic polymerization because of uncontrolled Michael-addition to the methacrylate backbone double bond. All other alkali metals lead to thermal runaway due to the fact that the exothermic reaction leads to an increased reaction rate which in turn leads to further exothermic reactions. This results in an undesired polymerization, which is why lithium salts are mainly used as (trans)esterification catalysts.

Of the lithium salts, lithium amide (LiNH₂) is most used for (trans)esterification. LiNH₂ is about six orders of magnitude more basic than the alkoxides, based on its pkₛ value. In addition, LiNH₂ has a surprisingly long catalytic lifetime due to its insolubility and slow decomposition in protic media under anhydrous conditions, releasing ammonia. As good as the catalyst's delayed decomposition and extremely strong basicity are, these properties cause some problems in industrial production. Ammonia released from the LiNH₂ catalyst decomposition reaction can form a hard ammonium carbamate solid in the presence of water and carbon dioxide. This ammonium carbamate solid formation occurs particularly in or on low temperature heat exchangers where water can condense. These solids continue to grow until flow paths are completely clogged and cleaning by maintenance is required. Preventing the presence of carbon dioxide to avoid this process is extremely difficult, as all (trans)esterification processes involve the use of polymerization inhibitors, especially hydrochinone monomethylether (HOME). These inhibitors require the presence of oxygen, usually by air or diluted air. Atmospheric carbon dioxide is introduced into the process with the air, which is usually purified, dried, and diluted if necessary, resulting in the carbamate solids mentioned earlier.

Among the formation of solids that plug vent lines and heat exchanger the insolubility in LiNH₂ has its own challenge and safety aspect. Unused or decomposed catalyst is floating as single particles within the reaction media. Due to polymer formation within the reaction media basically all MMA transesterification catalyst particles are preferably filtered off, after the reaction is finished, during transfer of the crude product from the reactor to a container and before the product is finally filtered and distilled. This leads to agglomerations of lithium amide particles in high concentration within a burnable mixture. The replacement and especially storage for disposal is therefore dangerous and can easily lead to fire and/or release of ammonia due to catalyst decomposition with atmospheric moisture. Additionally, the catalyst particles tend to agglomerate in valves, recirculation lines and especially in spray nozzles which therefore reliably plug and require maintenance.

Among all the disadvantages of LiNH₂, the most serious is the previously touted alkalinity of the catalyst. The use of LiNH₂ as a catalyst results in a significant amount of the crude product undergoing multiple Michael-addition reaction to the MMA backbone, ending up in a non-volatile oligomer. For distilled products, this means a complete loss of raw material, which cannot be reused or recovered, and a reduction in reaction yield. Nevertheless, these oligomers can be invisible to analytical methods such as gas chromatography (GC). However, their presence reduces the distillation yield. Unfortunately, the amount present is difficult to quantify, usually only by nuclear magnetic resonance (NMR), gel permeation chromatography (GPC), or high-pressure liquid chromatography (HPLC).

To overcome the safety concern of LiNH₂ containing filter residues, the maintenance of plugging and cleaning of heat exchanger the utilization of lithium methoxide (LiOMe) seems highly beneficial. LiOMe is the equivalent of the intermediate species released after (trans)esterification. Therefore, use of LiOMe would not introduce a foreign species into the process and would be the cleanest approach of catalysis.

Unfortunately, the solubility of lithium alkoxides, especially methanolate, proves to be a decisive disadvantage in large-scale applications. While LiNH₂ releases only a fraction of the catalytic power over a long period of time, the full catalytic efficiency of lithium alcoholates is available immediately at the start of the reaction. What sounds like a real advantage turns out to be a process engineering problem in production. The initial phase of an industrial-scale (trans)esterification reaction is compromised by a high methanol concentration in the reactor. The distillation apparatus must first be aligned to achieve a specified methanol purity before the methanol is removed from the reaction system. To accomplish this, a large portion of the condensed methanol returns to the reaction system to be purified again via distillation to increase purity. Unfortunately, the presence of a high catalyst and a high methanol concentration strong favors the formation of Michael-addition products that form methoxyisobutyric acid esters. These not only represent a loss of yield, but are also volatile and enter the final product, where they usually cause the material to fail to meet the purity specification.

US20200331845 describes a process for preparing dimethylaminoalkyl (meth)acrylates from alkyl (meth)acrylate and dimethylaminoalkanol. It relates to the use of a catalyst system comprising a solution of a lithium alkoxide in alcohol in the preparation of a dimethylaminoalkyl (meth)acrylate.

WO2005058862 describes a process for the preparation of glycerol carbonate methacrylate in the presence of metal chelate catalysts of the metal ion 1,3-diketonate type, in particular zirconium acetylacetonate. “V4” from the examples shows for lithium methylate the massive formation of 20 wt.-% high boilers and how difficult it is to run a transesterification reaction with bases.

US2013172598 describes a method for producing ethylene glycol dimethacrylate, wherein lithium amide (LiNH₂) and lithium chloride (LiCl) is used as catalyst. Comparative example 2 shows the usage of LiOMe as a negative example, which does not work for transesterification reactions.

Therefore, the object of the present invention is to provide a method that overcomes the aforementioned problems when using strong basic catalysts.

### SUMMARY OF THE INVENTION

The invention is a method for preparing an alkyl (meth)acrylate product by a (trans)esterification reaction of a reaction mixture in a reactor system, the reactor system comprising a reboiler, a reaction chamber comprising the reaction mixture, a column with a column head, a vapor transfer line, a condenser, a reflux tank, a reflux line, a distillate take off line, and a receiver vessel,
wherein the reaction mixture comprises a (meth)acrylate starting material and a first alcohol which are converted by the (trans)esterification reaction at a reaction temperature and a given pressure in the reaction chamber in the presence of a strong basic catalyst into the alkyl (meth)acrylate product and a side product,
wherein over a predominant amount of time during the (trans)esterification reaction at least a portion of the side product is continuously removed by distillate take off, and
wherein over a predominant amount of time during the (trans)esterification reaction the given pressure is repeatedly adjusted in order to maintain a range of the reaction temperature.

The present invention carries out the reaction under significant lower temperature. The reaction temperature in the reactor is kept constant by repeatingly adjusting the pressure in the reaction chamber to the changing boiling temperature of the reaction mixture. Running the reaction under vacuum or reduced pressure allows in addition the removal of methanol.

It was surprisingly found that the formation of methoxyisobutyric ester can be avoided if the reaction is carried out under this significantly lower temperature although strong basic catalysts, like LiOMe, are used and despite the presence of high concentrations of methanol.

Furthermore, with sufficiently low temperature, there is no loss in activity of the strong basic catalysts as the catalysts are active at room temperature. The filtration of the process liquid is improved enormously, so that significantly fewer filters need to be replaced and cleaned. In addition, the color of the crude product, that is the product before filtration and distillation, improves from amber brown to light yellow, indicating an increased purity of the product. The distillation of the new process material is running much longer with less steam pressure increase on wiped film evaporators per same product amount since the wall and waste tubes are less plugged.

An additional advantage of the invention is that by regulating the temperature through pressure adjustment thereby avoiding a temperature threshold being exceeded in the reactor before the end of the reaction, less excess (meth)acrylate starting material is needed in the reaction chamber for reaction temperature control. Reducing excess amounts of the (meth)acrylate starting material allows the amount of the first alcohol to be increased instead. In this way, the reaction chamber volume can be used more efficiently with regard to the space-time yield.

### DESCRIPTION OF THE DRAWINGS

FIG. 1 illustrates a reactor system according to the invention, the reactor system comprises a reboiler 12, a reaction chamber 1, a column 2 with a column head 3, a vapor transfer line 4, a condenser 5, a reflux tank 6, a reflux line 7, a distillate take off line 8 with a mass flow meter 10, a receiver vessel 9, and a recycle line 11.

### DETAILED DESCRIPTION OF THE INVENTION

Unless otherwise particularly defined herein, the related terms used in the present invention have the following definitions.

As used herein, the term “reactor system” refers to a reactor in which a chemical reaction of a reaction mixture can take place, and wherein the contents of the reactor can be subjected to a distillation process before the course of a chemical reaction and/or during the course of a chemical reaction and/or after the course of a chemical reaction or independently of a chemical reaction. The advantage of such a reactor is that it is not necessary to provide several chambers, one for a reaction to take place and one for the substance or substances to be distilled, thus saving material, time for a transfer of the substances and costs. The reactor system comprises at least a reboiler, a reaction chamber, a column with a column head, a feed line to the reaction chamber or to the column, a vapor transfer line, a condenser, a reflux tank, a reflux line, a distillate take off line, and a receiver vessel.

As used herein, the term “(trans)esterification” or “(trans)esterification reaction” refers to both an esterification reaction and a transesterification reaction. Or it refers to either an esterification reaction or a transesterification reaction depending on the respective context.

As used herein, the term “(meth)acrylate” refers to both (meth)acrylic acid and (meth)acrylic acid ester. Furthermore, it refers to both methacrylate and acrylate. Or it refers to methacrylate or acrylate depending on the respective context. It also refers to methacrylic acid and methacrylic acid ester. For example, the term “a (meth)acrylate compound” refers to (meth)acrylic acid and a (meth)acrylic acid ester, e.g. an alkyl (meth)acrylate.

As used herein, the term “starting material” refers to raw material, initial material, educts, feedstock, reactants or initial reactants which can be used to undergo a chemical reaction, whereby the chemical reaction may result at least in a product or products, which can include side-products or by-products.

As used herein, the term “reboiler” refers to an apparatus for heating a liquid and/or converting a liquid to its vapor or, in other words, gaseous state.

As used herein, the term “reaction chamber” refers to a container in which a chemical reaction is carried out. There are a wide variety of chambers being referred to as such. For example, the sizes of reaction chambers range from micro reaction chambers, which hold a few microliters, to reaction chambers for a few milliliters, to chambers with a volume of numerous cubic meters. The most important characteristic of each reaction chamber is its resistance to the reaction conditions.

As used herein, the term “reaction mixture” refers to a mixture of substances that participate in a reaction. Such a reaction mixture can comprise or consist of:
the starting material(s),
the starting material(s) and possible additive(s) such as auxiliary substances, which can be, for example, catalysts or other reaction accelerators,
the starting material(s) and product(s) including possible side-product(s),
the starting material(s), additive(s) and product(s) including possible side-product(s),
the product(s) including possible side-product(s) and the additive(s),
the product(s) alone and additive(s),
the product(s) including possible side-product(s), or
the product(s) alone.

As used herein, the term “column” refers to an apparatus for the thermal separation of mixtures. To avoid heat loss, the column can be an insulated, preferably cylindrical, tube, which can be made in particular of steel, high-alloy stainless steels, glass or plastic. The height of the column body can mainly be dictated by the required quality of separation; the diameter by the volume flow of the mixture to be separated. The column can be placed between the reaction chamber and the distillation head. The number of individual distillations required for the same separation performance can also be referred to as the “theoretical plate number”. At the surface of the column, the equilibrium between the liquid and gaseous phases can constantly be re-established by condensation and evaporation. As a result, the proportion of the low-boiling component continues to increase towards the top, while the higher-boiling component flows back into the reaction chamber, the sump. The size of the surface area of the column can be greatly increased in various ways by the design of trays, as in the Vigreux column, or by filling with packing or structured packing.

As used herein, the term “feed line” refers to supply lines which feed substances or substance mixtures to e.g. the reaction chamber or to the column.

As used herein, the term “condenser” refers to a unit in which the vapor produced during distillation, which can be composed of the various volatile components of the solution to be separated, can liquefy by cooling.

As used herein, the term “reflux tank” refers to a container into which condensed distillate flows, which then either flows back to the column and/or into the reaction chamber, or is removed from the system.

As used herein, the term “reflux line” refers to a line which can feed distillate from the reflux tank back to the column or to the reaction chamber.

As used herein, the term “distillate take off line” refers to a line which can remove at least a portion of distillate from the reflux tank, or in general from the reactor system, or the distillation system.

As used herein, the term “pressure stages” refers to pressure drop or pressure build-up at defined pressure values. The difference between two pressure stages can be e.g. less than 1000 mbar, less than 500 mbar, less than 100 mbar, less than 50 mbar, or less than 10 mbar.

As used herein, the term “recipe control” refers to a control system which, after evaluation of certain data such as the temperature of the reaction chamber, can automatically control certain processes such as pressure adjustment.

As used herein, the term “wt.-%” refers to weight percentage.

As used herein the term “sparkler filter” refers to a filter where unfiltered liquid is fed under positive pressure into a filter. The liquid reaches the opening on sides of the filter plates. Suspended particles are retained on the filter media resting on the filter plate. Clean filtrate emerges out from the peripheral holes of the plate, into the tank and comes out from the bottom outlet. The filtration in the sparkling filter is carried out until the holding capacity of the cake is reached or until the rate of filtrate flow becomes too slow.

The problem underlying the present invention is solved by a method for preparing an alkyl (meth)acrylate product by a (trans)esterification reaction of a reaction mixture in a reactor system, the reactor system comprises a reboiler, a reaction chamber comprising the reaction mixture, a column with a column head, a vapor transfer line, a condenser, a reflux tank, a reflux line, a distillate take off line, and a receiver vessel, the reaction mixture comprises a (meth)acrylate starting material and a first alcohol which are converted by the (trans)esterification reaction at a reaction temperature and a given pressure in the reaction chamber in the presence of a strong basic catalyst into the alkyl (meth)acrylate product and a side product, wherein over a predominant amount of time during the (trans)esterification reaction at least a portion of the side product is continuously removed by distillate take off, and wherein over a predominant amount of time during the (trans)esterification reaction the given pressure is repeatedly adjusted in order to maintain a range of the reaction temperature.

In preferred embodiments of the invention the (meth)acrylate starting material is methyl methacrylate and the first alcohol is ethylene glycol or hydroxyethyl methacrylate, and wherein the product is ethylene glycol dimethacrylate.

According to the invention the strong basic catalyst preferably comprises one or more compounds selected from the group consisting of alkali oxides, earth alkali oxides, alkali hydroxides, earth alkali hydroxides, alkali alkoxides, earth alkali alkoxides, alkali amides, and earth alkali amides.

More preferably the strong basic catalyst used in the method of the invention comprises one or more compounds selected from the group consisting of calcium oxide (CaO), calcium hydroxide (Ca(OH)₂), lithium hydroxide (LiOH), sodium methanolate (NaOMe), lithium methanolate (LiOMe), lithium tert-butoxide (LiOt-Bu), lithium iso-propoxide (LiOiPr), and lithium amide (LiNH₂), preferably wherein the strong basic catalyst comprises one or more compounds selected from the group consisting of lithium methanolate (LiOMe) and lithium amide (LiNH₂), and most preferably wherein the strong basic catalyst comprises or is lithium methanolate (LiOMe).

The method according to the invention has the advantage that catalysts previously unsuitable or poorly suited for the (trans)esterification reaction can now be used efficiently in the (trans)esterification process, which leads to an accelerated reaction speed and to an increased space-time yield.

The reactor system may further comprise one or more elements selected from the group of one or more mass flow meters, a recycle line, and a feed line to the reaction chamber or to the column, preferably wherein the feed line is a first feed line, and wherein the reactor system further comprises a second feed line to the reaction chamber or to the column, and, optionally, further feed lines to the reaction chamber or to the column.

Preferably, the feed line is a first feed line, and the reactor system comprises a second feed line to the reaction chamber or to the column, and, optionally, further feed lines to the reaction chamber or to the column. This has the advantage that substances and/or mixtures can be added to the reaction chamber and/or column via several feed lines. Substances and/or mixtures can be added simultaneously or successively and/or continuously or discontinuously via these feed lines. Different substances and/or mixtures can be added to the reaction chamber and/or column via the feed lines. Also, identical substances and/or mixtures can be added to the reaction chamber and/or column via the feed lines. Furthermore, identical substances and/or mixtures and different substances and/or mixtures can be added to the reaction chamber and/or column via the feed lines. These substances or mixtures can be, for example, the starting materials used for the reaction and/or additives such as reaction accelerators, reaction inhibitors, buffer, solvents, stabilizer, water, viscosity index improvers, thickeners, antioxidants, corrosion inhibitors, dispersants, high pressure additives, defoamers, catalysts or enzymes.

Through the reflux line distillate can flow back onto the column or into the reaction chamber. The reflux tank stores reflux of the distillate.

The first alcohol can be a linear C₂-C₆ alkanol, a linear C₂-C₆ alkandiol, a linear vicinal C₂-C₆ alkandiol, or, preferably, ethylene glycol.

In the method according to the invention the (meth)acrylate starting material may comprise methyl (meth)acrylate, and the side product may comprise methanol, or the (meth)acrylate starting material may comprise ethyl (meth)acrylate, and the side product may comprise ethanol, or the (meth)acrylate starting material may comprise n-butyl (meth)acrylate and the side product may comprise butanol, or the (meth)acrylate starting material may comprise (meth)acrylic acid, and the side product may comprise water, preferably the (meth)acrylate starting material comprises methyl (meth)acrylate, and the side product comprises methanol.

In the inventive method the side product can also be removed in form of a mixture of the side product and the (meth)acrylate starting material.

When performing the method according to the invention, it is a preferred option to compensate at least a portion of the converted (meth)acrylate starting material by adding a further amount of the (meth)acrylate starting material to the reaction mixture, preferably via a feed line to the reaction chamber or to the column.

When performing the method according to the invention, it is a further preferred option that a filling level of the reaction chamber is kept constant by compensating at least a portion of the converted (meth)acrylate starting material and/or of the first alcohol by adding a further amount of the (meth)acrylate starting material and/or of the first alcohol to the reaction mixture in the reaction chamber, preferably via a feed line to the reaction chamber or to the column.

This has the advantage that available volume in the reactor chamber is used for the (trans)esterification reaction, which increases the space-time yield. Preferably further (meth)acrylate starting material is added to the maximal reactor filling level to use the maximal volume of the reactor for the (trans)esterification reaction and to even more increase the space-time yield. This has the further advantage that an optimal heat transfer from the outer wall into the reaction wall into the reaction medium is achieved as a maximum utilized heat transfer area is provided. Further preferably, the reactor filling level is maintained constant during the (trans)esterification process.

Preferably the (trans)esterification reaction is carried out in the presence of a polymerization inhibitor.

This has the advantage that polymerization is inhibited, and the space-time yield is increased. Furthermore, the product can be purer. The polymerization inhibitor HQME can be present in the range of 1 ppm to 1000 ppm, preferably 10 to 1000 ppm, more preferred 10 to 500 ppm, and most preferred 50 to 100 ppm, with ppm in weight. The polymerization inhibitor Tempol or any of its derivatives can be present in the range of 0.1 ppm to 1000 ppm, more preferred 1 to 500 ppm, and most preferred 10 to 100 ppm, with ppm in weight.

As an inhibitor p-phenylenediamines, phenothiazine and hydroxylamines like N,N-bis(2-hydroxypropyl)hydroxylamine (HPHA) and N,N-diethylhydroxylamine (DEHA) can be used. Most preferred inhibitor are hydrochinone (HQ), its methyl ether HQME and 2,2,6,6-tetramethylpiperidinyloxyl (TEMPO), or its derivatives like 4-hydroxy-2,2,6,6-tetramethylpiperidinyloxyl (TEMPOL) and any derivative of this functionalizing the hydroxy group like methacrylic ester of 4-hydroxy-2,2,6,6-tetramethylpiperidinyloxyl.

Preferably the (trans)esterification reaction is carried out with introduction of oxygen into the reaction mixture.

Beside oxygen also air can be introduced into the reaction to speed up the reaction process. The oxygen may be introduced continuously or discontinuously, preferably the oxygen is introduced continuously.

Preferably, the repeated adjustment of the given pressure is performed in pressure stages, preferably wherein the difference between two pressure stages is less than 500 mbar, preferably less than 100 mbar, more preferably less than 50 mbar, and even more preferably less than 10 mbar.

This has the advantage that the pressure can be adjusted in steps which can gradually adjust the pressure. Furthermore, through pressure steps it is possible to adjust the pressure in reaction to the situation in the reactor. The lower the pressure stages are, the more sensitive the reaction can be.

A pressure drop is favorable for separation of a side product.

Preferably, the (trans)esterification reaction is started at a pressure within the range of 0.1 bar to 5.0 bar, preferably 0.1 bar to 4.0 bar, more preferably 0.1 bar to 3.0 bar, even more preferably 0.1 bar to 2.0 bar, most preferably 0.5 bar to 1.5 bar, for example 1 bar, or wherein the (trans)esterification reaction is started at a pressure of above 1.0 bar and the reaction process ends at a pressure of below 1.0 bar. If side-product formation is significantly increased at elevated temperature, it is advisable to allow the reaction to end at a pressure below normal pressure, i.e. normal pressure can be defined as 1 bar, in order to lower the reaction temperature above the boiling temperature of the reactants in a targeted manner.

Preferably, the repeated adjustment of the given pressure is performed continuously.

This has the advantage that the pressure can be controlled and adjusted when the reaction temperature is lower than the boiling temperature. Shutdowns of the reactor due to boiling of the reaction mixture can be prevented.

As the boiling temperature of the reaction mixture changes during the (trans)esterification reaction, preferably the (trans)esterification reaction is carried out while the pressure is repeatedly adjusted to the boiling temperature of the reaction mixture. The regulation of pressure stages can also be affected automatically by a recipe control.

This has the advantage that the reaction conditions remain more or less constant over a predominant amount of time or the whole time of the (trans)esterification reaction and that the reaction conditions remain at an optimal reaction temperature. This has the further advantage that the reaction is conducted at optimal speed and polymerization as well as further side effects as generation of undesirable side products including in particular the Michael addition to the methacrylic acid derivatives by the corresponding alcoholates as nucleophiles are reduced to a minimum.

Within the present invention the given pressure is repeatedly adjusted in a way to maintain the reaction temperature typically in the range of from 70° C. to not more than 130° C. Preferably, the given pressure is repeatedly adjusted in a way to maintain the reaction temperature in the range of from 80° C. to not more than 125° C., more preferably in the range of from 90° C. to not more than 120° C.

The starting material comprises the (meth)acrylate and the first alcohol. The reaction mixture may comprise two or more starting materials and two or more products. Further preferably, the two or more starting materials are a first educt, a second educt, and, optionally, one or more further compounds, and wherein the two or more products are a first product, a second product, and, optionally, one or more further products.

The reaction mixture can further comprise additives such as reaction accelerators, reaction inhibitors, buffer, solvents, stabilizer, water, viscosity index improvers, thickeners, antioxidants, corrosion inhibitors, dispersants, high pressure additives, defoamers, catalysts or enzymes.

Preferably the catalyst is a strong basic catalyst. Stabilizers comprise e.g. HQME, phenotiazin (PTZ), Tempol, or hydroxytempol. Furthermore, at least an alcohol, e.g. a second alcohol, is formed as a side product. Further side products can be formed by the reaction, which then are also present in the reaction mixture.

As mentioned above an additional advantage of the invention is that less excess (meth)acrylate starting material is needed in the reaction chamber for reaction temperature control. When performing the method according to the invention the ratio of the first alcohol to the (meth)acrylate starting material before the start of the reaction can be in the range of 1:1 to 1:5. It is preferred that the ratio is in the range of 1:1 to 1:2, more preferably in the range of 1:1.1 to 1:1.5, and most preferably in the range of 1:1.1 to 1:1.3, for example 1:1.2.

The pressure change in the reaction chamber is performed by actively changing a gas space or liquid-filled space. The gas for pressure regulation can be oxygen, nitrogen, ethylene oxide or isobutene or mixtures thereof. Increasing the pressure by a liquid is also possible, but it limits the reactor volume and is therefore disadvantageous. The use of a gas has therefore proved to be preferred. Since air must always be supplied to the reaction for stabilization, this gas is most preferably an oxygen-containing gas such as air or diluted air. If the reactant is also a gas, it has proved particularly preferable to mix this reactant with oxygen or an oxygen-containing gas.

Preferably the reboiler is an internal or external heating coil, a heating jacket, an internal heat exchanger or an external heat exchanger.

The following examples aim to explain and clarify to the skilled person the principles of the present invention and to show how the invention can be applied in practice.

### EXAMPLES

### Comparative Example 1

### Generation of EGDMA Using Lithium Amide (LiNH₂) as a Catalyst

4 263 kg of hydroxyethyl methacrylate (HEMA), 4 234 kg of methyl methacrylate (MMA), with 0.183 kg HQME as inhibitor and 8 kg lithium amide as catalyst are combined in a stirred tank reactor, which is a stirred reaction vessel in a reactor 1, provided with agitator, a feed line 13, reboiler 12, distillation column 2 and condenser 5 and stirred while passing in air. To stabilize the column 2, a total of 22.68 kg/h of MMA containing 0.1 wt.-% of hydroquinone monomethyl ether and 0.01 wt.-% of 4-hydroxy-2,2,6,6-tetramethylpiperidin-1-oxyl in dissolved form are introduced into the column runback. The reactor 1 is heated until distillate flow is obtained, with the column initially being operated with total reflux at 40.8 kg/min distillate flow. As soon as the methanol concentration at the top of the column increases to 75 wt.-%, the methanol/MMA mixture is taken off to that extend that the concentration is maintained at 75 wt.-%, or at least 1.63 kg/min are taken off. The MMA stock in the reactor is supplemented by metered addition of equal parts of MMA per part of methanol/MMA mixture taken off. A total of 3039 kg of MMA are thus introduced over the reaction period and no more replacement is done once the total is obtained. A further catalyst dosage than the initial is not done. The reaction temperature raises during the reaction to about 132° C. The reaction is complete when the residual HEMA has an amount of less than 0.8 wt.-%, without MMA as part of integration, with a good constant total time of 20 h. Once the reaction is completed, the excess MMA is taken off under reduced pressure, with the pressure gradually being reduced to 0.042 bar. When no more MMA distills off or the ethylene glycol dimethacrylate (EGDMA) concentration in the distillate exceeds a threshold of 35 wt.-% and the vacuum is broken. The contents of the tank, comprising the catalyst and ethylene glycol dimethacrylate, are freed of catalyst with the aid of a sparkler filter.

### Results:

In this example 6486 kg of EGDMA crude containing 92.4 wt.-% EGDMA, 0.8 wt.-% HEMA, 0.8 wt.-% MMA, 0.4 wt.-% diethylene glycol dimethacrylate, 0.24 wt.-% Michael-addition product of EGDMA and methanol, 1.8 wt.-% Michael-addition product of EGDMA and HEMA are obtained resulting after distillation in 98.2 wt.-% EGDMA, 0.8 wt.-% HEMA, 0.7 wt.-% MMA, 0.2 wt.-% diethylene glycol dimethacrylate.

### Comparative Example 2

### Generation of EGDMA Using Lithium Methoxide (LiOMe) as a Catalyst

4 263 kg of HEMA, 4 234 kg of methyl methacrylate (MMA), with 0.183 kg HQME as inhibitor and 13.6 kg lithium methoxide as catalyst are combined in a stirred tank reactor, which is a stirred reaction vessel in a reactor 1, provided with agitator, a feed line 13, reboiler 12, distillation column 2 and condenser 5 and stirred while passing in air. To stabilize the column 2, a total of 22.68 kg/h of MMA containing 0.1 wt.-% of hydroquinone monomethyl ether and 0.01 wt.-% of 4-hydroxy-2,2,6,6-tetramethylpiperidin-1-oxyl in dissolved form are introduced into the column runback. The reactor 1 is heated until distillate flow is obtained, with the column initially being operated with total reflux at 40.8 kg/min distillate flow. As soon as the methanol concentration at the top of the column increases to 75 wt.-%, the methanol/MMA mixture is taken off to that extend that the concentration is maintained at 75 wt.-%, or at least 1.63 kg/min are taken off. The MMA stock in the reactor 1 is supplemented by metered addition of equal parts of MMA per part of methanol/MMA mixture taken off. A total of 3039 kg of MMA are thus introduced over the reaction period and no more replacement is done once the total is obtained. A further catalyst dosage than the initial is not done. The reaction temperature raises during the reaction to about 132° C. The reaction is complete when the residual HEMA has an amount of less than 0.8 wt.-%, without MMA as part of integration, with a good constant total time of 20 h. Once completed the excess MMA is taken off under reduced pressure, with the pressure gradually being reduced to 0.042 bar. When no more MMA distills off or the EGDMA concentration in the distillate exceeds a threshold of 35 wt.-%, the vacuum is broken. The contents of the tank, comprising the catalyst and ethylene glycol dimethacrylate, are freed of catalyst with the aid of a sparkler filter.

### Results:

This gives 6486 kg of EGDMA crude: containing 87.5 wt.-% EGDMA, 0.93 wt.-% HEMA 0.6 wt.-% MMA, 0.4 wt.-% diethylene glycol dimethacrylate, 2.73 wt.-% Michael-addition product of EGDMA and methanol, 5.15 wt.-% Michael-addition product of EGDMA and HEMA resulting after distillation in 96.2 wt.-% EGDMA, 0.9 wt.-% HEMA, 0.44 wt.-% MMA, 0.16 wt.-% diethylene glycol dimethacrylate, 2.0 wt.-% Michael-addition product of EGDMA and methanol.

### Comparative Example 3

### Generation of EGDMA Using LiNH₂ as a Catalyst

2 038 kg of ethylene glycol, 4 927 kg of methyl methacrylate (MMA), with 0.183 kg HQME as inhibitor and 8.165 kg lithium amide as catalyst are combined a stirred tank reactor, which is a stirred reaction vessel in a reactor 1, provided with agitator, a feed line 13, reboiler 12, distillation column 2 and condenser 5 and stirred while passing in air. To stabilize the column 2, a total of 22.68 kg/h of MMA containing 0.1 wt.-% of hydroquinone monomethyl ether and 0.01 wt.-% of 4-hydroxy-2,2,6,6-tetramethylpiperidin-1-oxyl in dissolved form are introduced into the column runback. The reactor 1 is heated until distillate flow is obtained, with the column initially being operated with total reflux at 40.8 kg/min distillate flow. As soon as the methanol concentration at the top of the column 3 increases to 75 wt.-%, the methanol/MMA mixture is taken off to that extend that the concentration is maintained at 75 wt.-%, or at least 1.63 kg/min are taken off. The MMA stock in the reactor 1 is supplemented by metered addition of equal parts of MMA per part of methanol/MMA mixture taken off. A total of 3039 kg of MMA are thus introduced over the reaction period and no more replacement is done once the total is obtained. A further catalyst dosage than the initial is not done. The reaction temperature raises during the reaction to about 132° C. The reaction is complete when the residual HEMA has an amount of less than 0.8 wt.-%, without MMA as part of integration, with a good constant total time of 22 h. Once completed the excess MMA is taken off under reduced pressure, with the pressure gradually being reduced to 0.042 bar. When no more MMA distills off or the EGDMA concentration in the distillate exceeds a threshold of 35 wt.-%, the vacuum is broken. The contents of the tank, comprising the catalyst and ethylene glycol dimethacrylate, are freed of catalyst with the aid of a sparkler filter.

### Results:

This gives 6486 kg of EGDMA crude: containing 92.5 wt.-% EGDMA, 1.22 wt.-% HEMA, 0.46 wt.-% MMA, 0.04 wt.-% diethylene glycol dimethacrylate, 0.14 wt.-% Michael-addition product of EGDMA and methanol, 2.17 wt.-% Michael-addition product of EGDMA and HEMA resulting after distillation in 98.2 wt.-% EGDMA, 0.99 wt.-% HEMA, 0.41 wt.-% MMA, 0.05 wt.-% diethylene glycol dimethacrylate, 0.19 wt.-% Michael-addition product of EGDMA and methanol.

### Example 1

### Generation of EGDMA Using LiOMe as a Catalyst and Adjustment of Pressure

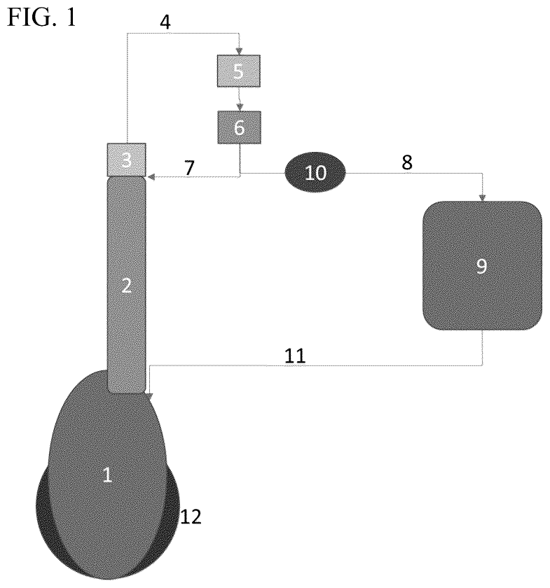

4 264 kg of HEMA, 4 235 kg of MMA, with 0.183 kg HQME as inhibitor and 13.6 kg lithium methoxide as catalyst are combined with reference to FIG. 1 in a stirred tank reactor, which is a stirred reaction vessel in a reactor 1, provided with agitator, a feed line 13, reboiler 12, distillation column 2 and condenser 5 and stirred while passing in air. To stabilize the column, a total of 22.68 kg/h of MMA containing 0.1 wt.-% of hydroquinone monomethyl ether and 0.01 wt.-% of 4-hydroxy-2,2,6,6-tetramethylpiperidin-1-oxyl in dissolved form are introduced into the column runback. The reactor is heated until distillate flow is obtained, with the column initially being operated with total reflux at 40.8 kg/min distillate flow. The reactor pressure is decrease by 0.007 bar whenever the reactor temperature got closer to the target setpoint of 100° C. than 0.5° C. If the temperature was not further away than 0.5° C., 2 minutes after a pressure decrease was done the pressure decrease is repeated. Once the temperature has been decreased, the pressure is increased if the reactor temperature was further away than 1° C. As soon as the methanol concentration at the top of the column increases to 75 wt.-%, the methanol/MMA mixture is taken off to that extend that the concentration is maintained at 75 wt.-%, or at least 1.63 kg/min are taken off. The MMA stock in the reactor is supplemented by metered addition of equal parts of MMA per part of methanol/MMA mixture taken off. A total of 3039 kg of MMA are thus introduced over the reaction period and no more replacement is done once the total is obtained. A further catalyst dosage than the initial is not done. The reaction temperature maintains well at 100° C. during the reaction, the ending pressure was 0.525 bar. The reaction is complete at the same time the reference batch in comparative example 2 is finished at a total time of 20 h. Once the reaction is completed the excess MMA is taken off under reduced pressure, with the pressure gradually being reduced to 0.042 bar. When no more MMA distills off or the EGDMA concentration in the distillate exceeds a threshold of 35 wt.-%, the vacuum is broken. The contents of the tank, comprising the catalyst and ethylene glycol dimethacrylate, are freed of catalyst with the aid of a sparkler filter.

### Results:

This gives 6486 kg of EGDMA crude: containing 95.1 wt.-% EGDMA, 1.5 wt.-% HEMA 0.33 wt.-% MMA, 0.4 wt.-% diethylene glycol dimethacrylate, 0.3 wt.-% Michael-addition product of EGDMA and methanol, 1.7 wt.-% Michael-addition product of EGDMA and HEMA-->resulting after distillation in 98.18 wt.-% EGDMA, 0.28 wt.-% MMA, 1.22 wt.-% HEMA, 0.13 wt.-% diethylene glycol dimethacrylate, and 0.13 wt.-% Michael-addition product of EGDMA and methanol.

### Example 2

### Generation of EGDMA Using LiOMe as a Catalyst and Adjustment of Pressure

2 038 kg of ethylene glycol, 4 927 kg of MMA, with 0.183 kg HQME as inhibitor and 13.6 kg lithium methoxide as catalyst combined in a stirred tank reactor, which is a stirred reaction vessel in a reactor 1, provided with agitator, a feed line 13, a reboiler 12, distillation column 2 and condenser 5 and stirred while passing in air. To stabilize the column 2, a total of 22.68 kg/h of MMA containing 0.1 wt.-% of hydroquinone monomethyl ether and 0.01 wt.-% of 4-hydroxy-2,2,6,6-tetramethylpiperidin-1-oxyl in dissolved form are introduced into the column runback. The reactor 1 is heated until distillate flow is obtained, with the column 2 initially being operated with total reflux at 40.8 kg/min distillate flow. The reactor pressure is decrease by 0.007 bar whenever the reactor temperature got closer to the target setpoint of 100° C. than 0.5° C. If the temperature was not further away than 0.5° C., 2 minutes after a pressure decrease was done the pressure decrease is repeated. Once the temperature has been decreased, the pressure is increased if the reactor temperature was further away than 1° C. As soon as the Methanol concentration at the top of the column increases to 75 wt.-%, the methanol/MMA mixture is taken off to that extend that the concentration is maintained at 75 wt.-%, or at least 1.63 kg/min are taken off. The MMA stock in the reactor is supplemented by metered addition of equal parts of MMA per part of methanol/MMA mixture taken off. A total of 3039 kg of MMA are thus introduced over the reaction period and no more replacement is done once the total is obtained. A further catalyst dosage than the initial is not done. The reaction temperature maintains well at 100° C. during the reaction, the ending pressure was 0.511 bar. The reaction is complete at the same time the reference batch in comparative example 3 is finished at a total time of 22 h. Once the reaction is completed the excess MMA is taken off under reduced pressure, with the pressure gradually being reduced to 0.042 bar. When no more MMA distills off or the EGDMA concentration in the distillate exceeds a threshold of 35 wt.-%, the vacuum is broken. The contents of the tank, comprising the catalyst and ethylene glycol dimethacrylate, are freed of catalyst with the aid of a sparkler filter.

### Result:

This gives 6486 kg of EGDMA crude: containing 95.0 wt.-% EGDMA, 1.6 wt.-% HEMA, 0.41 wt.-% MMA, 0.04 wt.-% diethylene glycol dimethacrylate, 0.1 wt.-% Michael-addition product of EGDMA and methanol, 1.5 wt.-% Michael-addition product of EGDMA and HEMA resulting after distillation in 98.08 wt.-% EGDMA, 0.28 wt.-% MMA, 1.32 wt.-% HEMA, 0.13 wt.-% diethylene glycol dimethacrylate, and 0.13 wt.-% Michael-addition product of EGDMA and methanol.

Thus, it has been demonstrated to overcome the above mentioned safety concern of LiNH₂ by using lithium alcoholates, preferably of lithium methoxide (LiOMe). Surprisingly, the disadvantages of LiOMe providing an immediate catalytic efficiency at the start of the reaction leading to e.g. a high methanol concentration in the reactor, could have been successfully suppressed by the selected decrease in the constant reaction temperature in combination and optionally with an additional stepwise decrease in pressure. With reference to the comparative example 2 and the examples 1 and 2, the yield of product EGDMA after distillation could have been significantly increase by 1.28 wt.-% resulting after distillation form 97.54 wt.-% up to 98.18 wt.-% EGDMA at a decrease reaction temperature up to 32° C.

1. A method, comprising:
preparing an alkyl (meth)acrylate product by a (trans)esterification reaction of a reaction mixture in a reactor system, the reactor system comprising a reaction chamber comprising the reaction mixture, a feed line, a reboiler, a column with a column head, a vapor transfer line, a condenser, a reflux tank, a reflux line, a distillate take off line, and a receiver vessel,
wherein the reaction mixture comprises a (meth)acrylate starting material and a first alcohol which are converted by the (trans)esterification reaction at a reaction temperature and a given pressure in the reaction chamber in presence of a strong basic catalyst into the alkyl (meth)acrylate product and a side product,
wherein the (meth)acrylate starting material is methyl methacrylate and the first alcohol is ethylene glycol or hydroxyethyl methacrylate, and wherein a product is ethylene glycol dimethacrylate,
wherein the strong basic catalyst comprises one or more compounds selected from the group consisting of calcium oxide (CaO), calcium hydroxide (Ca(OH)₂), lithium hydroxide (LiOH), sodium methanolate (NaOMe), lithium methanolate (LiOMe), lithium tert-butoxide (LiOt-Bu), and lithium iso-propoxide (LiOiPr),
wherein over a predominant amount of time during the (trans)esterification reaction at least a portion of the side product is continuously removed by distillate take off, and
wherein over a predominant amount of time during the (trans)esterification reaction the given pressure is repeatedly adjusted in order to maintain a range of the reaction temperature,
wherein the repeated adjustment of the given pressure is performed in pressure stages, and
wherein the given pressure is repeatedly adjusted in a way to maintain the reaction temperature in a range of from 70° C. to not more than 130° C.

2. The method according to claim 1, further comprising:
removing the side product in form of a mixture of the side product and the (meth)acrylate starting material.

3. The method according to claim 1, further comprising:
compensating at least a portion of a converted (meth)acrylate starting material by adding a further amount of the (meth)acrylate starting material to the reaction mixture.

4. The method according to claim 1, further comprising:
carrying out the (trans)esterification reaction in presence of a polymerization inhibitor.

5. The method according to claim 1, further comprising:
carrying out the (trans)esterification reaction with introduction of oxygen into the reaction mixture.

6. The method according to claim 1, further comprising:
continuously performing a repeated adjustment of the given pressure.

7. The method according to claim 1, wherein the strong basic catalyst is lithium methanolate (LiOMe).

8. The method according to claim 1, wherein the repeated adjustment of the given pressure is performed in pressure stages, and wherein the difference between two pressure stages is less than 10 mbar.

9. The method according to claim 1, wherein the given pressure is repeatedly adjusted in a way to maintain the reaction temperature in a range of from 90° C. to not more than 120° C.

10. The method according to claim 3, further comprising:
adding a further amount of the (meth)acrylate starting material to the reaction mixture via a feed line to the reaction chamber or to the column.
